(19) United States
(12) Patent Application Publication
(10) Pub. No.: US 2024/0130611 A1
(43) Pub. Date: Apr. 25, 2024

(54) SYSTEM AND METHOD FOR AIRWAY INTUBATION USING WIRELESS TRANSMISSION OF MULTIPLE VIDEOS

(71) Applicants: Gentex Corporation, Zeeland, MI (US); TurningMode, LLC, Novelty, OH (US)

(72) Inventors: Eric P. Bigoness, Ada, MI (US); Jorge Zapata, Novelty, OH (US)

(21) Appl. No.: 18/491,984

(22) Filed: Oct. 22, 2023

Related U.S. Application Data
(60) Provisional application No. 63/380,468, filed on Oct. 21, 2022.

(51) Int. Cl. A61B 1/06; A61B 1/00; A61B 1/267

(52) U.S. Cl. CPC A61B 1/0684; A61B 1/00009; A61B 1/00045; A61B 1/267

(57) ABSTRACT

An intubation system is provided including an endoscope, a laryngoscope, and a display adaptor. The endoscope includes an endoscope video camera and at least one light emitting diode at a distal end, the endoscope video camera generating an endoscope video stream. The laryngoscope includes: a laryngoscope video camera for generating a laryngoscope video stream and a button. The display adapter is configured to receive the endoscope video stream and the laryngoscope video stream and to supply the video stream to a display. The button allows user selection of which of the laryngoscope video stream, and the endoscope video stream, or both video stream to supply from the display adaptor to the display.

### CROSS-REFERENCE TO RELATED APPLICATION

This application claims priority under 35 U.S.C. § 119(e) upon U.S. Provisional Patent Application No. 63/380,468, entitled “MULTI VIDEO SOURCE, WIRELESS SYSTEM, AND METHOD FOR AIRWAY INTUBATION” filed on Oct. 21, 2022, by Eric P. Bigoness et al., the entire disclosure of which is incorporated herein by reference.

### BACKGROUND OF THE DISCLOSURE

The present disclosure generally relates to an intubation system, and more specifically to an intubation system allowing use of both an intubation tube and a laryngoscope.

### SUMMARY OF THE DISCLOSURE

According to one aspect of the present disclosure, an intubation system is provided that includes: an endoscope, a laryngoscope, and a display adaptor. The endoscope includes an endoscope video camera and at least one light emitting diode at a distal end, the endoscope video camera generating an endoscope video stream. The laryngoscope includes: a laryngoscope video camera for generating a laryngoscope video stream, and a button. The display adapter is configured to receive the endoscope video stream and the laryngoscope video stream and to supply the video stream to a display. The button allows user selection of which of the laryngoscope video stream and the endoscope video stream, or both video streams to supply from the display adaptor to the display.

According to another aspect of the present disclosure, an intubation system is provided that includes an endoscope, a laryngoscope, and a display adaptor. The endoscope includes: an endoscope video camera and at least one light emitting diode at a distal end and an endoscope wireless video transmitter on the proximal end for receiving an endoscope video stream from the endoscope video camera and transmitting an endoscope wireless video broadcast. The laryngoscope includes: a laryngoscope video camera and a laryngoscope wireless video transmitter for receiving a laryngoscope video stream from the laryngoscope video camera and transmitting a laryngoscope wireless video broadcast. The display adapter is configured to receive at least one of the endoscope wireless video broadcast and the laryngoscope wireless video broadcast and to supply the video stream transmitted within the wireless video broadcasts to a display.

According to yet another aspect of the present disclosure, an intubation system includes an endoscope and a laryngoscope in communication with the endoscope. The endoscope includes an endoscope video camera and at least one light emitting diode at a distal end, the endoscope video camera generating an endoscope video stream. The laryngoscope includes: a laryngoscope video camera for generating a laryngoscope video stream; a button; and a laryngoscope display for displaying at least the laryngoscope video stream. The button provides user selection of which of the laryngoscope video stream and the endoscope video stream to display of the laryngoscope display.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a perspective view of an intubation system according to a first embodiment;

FIG. 2 is a perspective view of a laryngoscope of the intubation system shown in FIG. 1;

FIG. 3 is a perspective view of an intubation tube of the intubation system shown in FIG. 1;

FIG. 4 is a perspective view of an endoscope of the intubation system shown in FIG. 1;

FIG. 5 is an electrical circuit diagram in block form showing a first configuration of the intubation system; and

FIG. 6 is an electrical circuit diagram in block form showing a second configuration of the intubation system.

The components in the figures are not necessarily to scale, emphasis instead being placed upon illustrating the principles described herein.

### DETAILED DESCRIPTION

The present illustrated embodiments reside primarily in combinations of method steps and apparatus components related to an intubation system. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

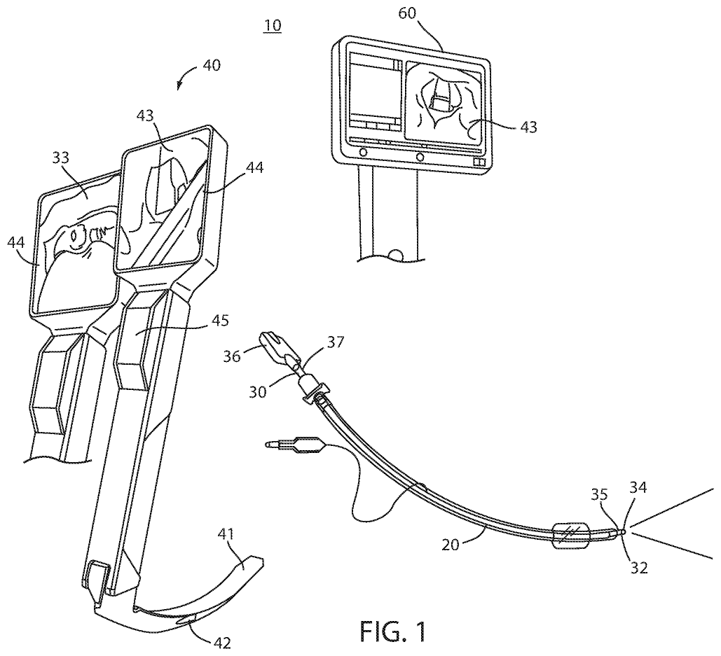

For purposes of description herein, the terms “upper,” “lower,” “right,” “left,” “rear,” “front,” “vertical,” “horizontal,” and derivatives thereof shall relate to the disclosure as oriented in FIG. 1. Unless stated otherwise, the term “front” shall refer to the surface of the element closer to an intended viewer, and the term “rear” shall refer to the surface of the element further from the intended viewer. However, it is to be understood that the disclosure may assume various alternative orientations, except where expressly specified to the contrary. It is also to be understood that the specific devices and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

Referring to FIGS. 1, 5, and 6, reference numeral 10 generally designates an intubation system. The intubation system 10 may include an intubation tube 20, an endoscope 30, a laryngoscope 40, and a display adaptor 50 for connection to a display 60. The endoscope 30 includes an endoscope video camera 32 and at least one light emitting diode 34 at a distal end 35 of the endoscope 30. The endoscope video camera 32 generates an endoscope video stream 33.

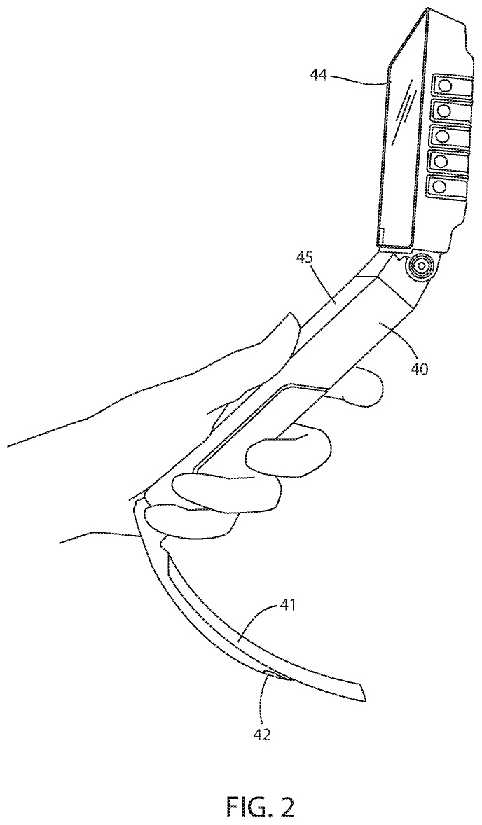

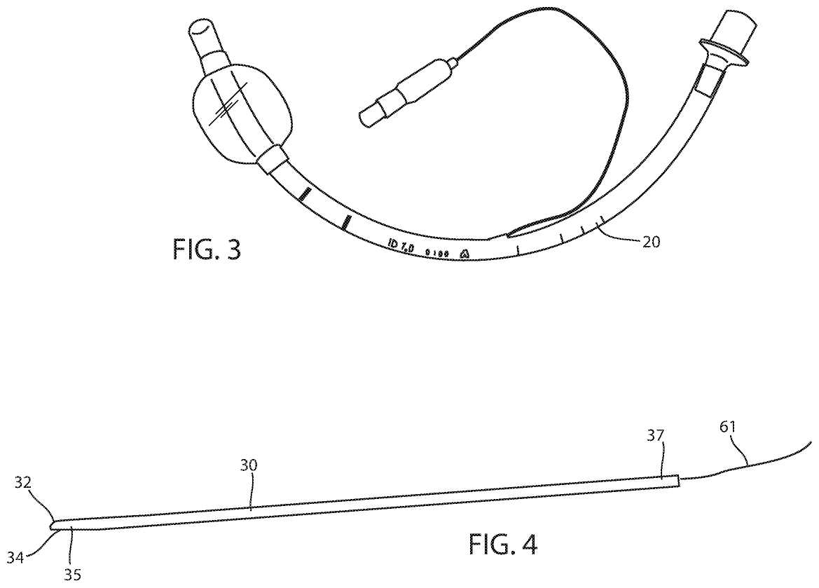

As shown in FIGS. 1 and 2, the laryngoscope 40 includes a laryngoscope video camera 42 for generating laryngoscope video stream 43. The laryngoscope 40 may further include a light source (not shown) such as one or more LEDs for illuminating the field of view of the video camera 42.

The display adapter 50 is configured to receive the endoscope video stream 33 and the laryngoscope video stream 43 and to supply one or both of the video stream to the display 60. A button 45 may provide user selection of which of the laryngoscope video stream 43, the endoscope video stream 33, or both video streams to supply from the display adaptor 50 to the display 60. The button 45 may be physical or virtual and may be provided on the laryngoscope 40 or at the display 60 via a touchscreen function.

The endoscope 30, the laryngoscope 40, and the display adaptor 50 may be connected by wires 61 (FIG. 4) for transmission of the video streams and any control signals or may be in wireless communication as discussed further below.

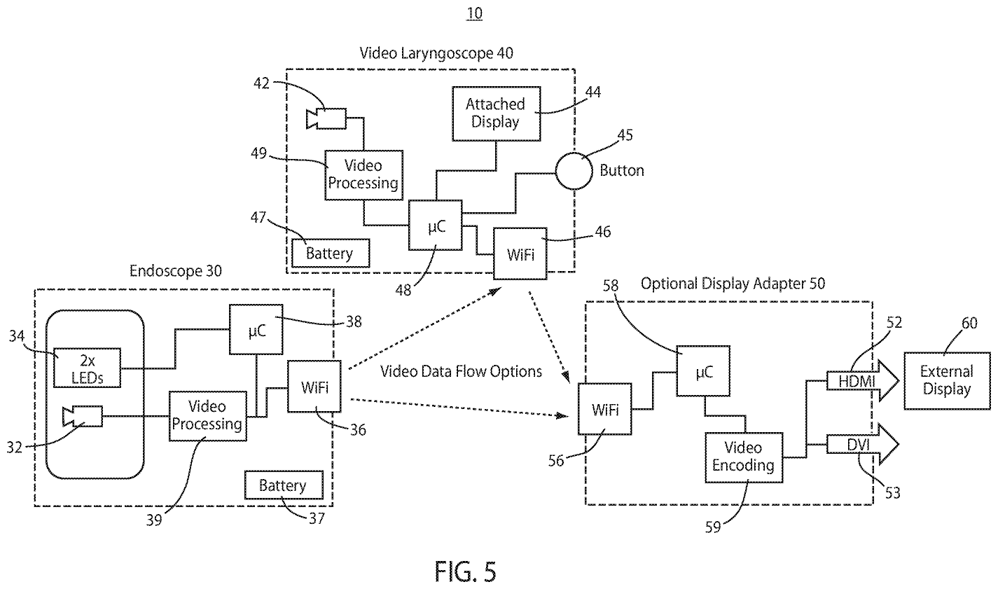

To provide for wireless communication, the endoscope 30 may further include an endoscope wireless video transmitter 36 (FIGS. 1 and 5) on the proximal end 37 of the endoscope 30 for receiving the endoscope video stream 33 from the endoscope video camera 32 and for transmitting an endoscope wireless video broadcast. The laryngoscope 40 may further include a laryngoscope wireless video transmitter 46 for receiving the laryngoscope video stream 43 from the laryngoscope video camera 42 and for transmitting a laryngoscope wireless video broadcast. The display adaptor 50 may include a wireless video receiver 56 for receiving the endoscope wireless video broadcast and/or the laryngoscope wireless video broadcast, and for supplying the video stream to the display 60.

The wireless transmission may be provided over an ad hoc, mesh, or point-to-point wireless network and may use an existing protocol like Wi-Fi to reduce costs with commodity parts and standard protocols. Security may be provided with a pairing process and Wi-Fi standard encryption. Bluetooth™ may be used as a secondary wireless data channel to aid in device discovery and video pairing negotiation. By providing wireless transmissions between the components of intubation system 10, wires 61 therebetween may be eliminated thereby reducing clutter. By reducing clutter, the need for the user to orchestrate each micro-maneuver to avoid tangling of wires is eliminated while also making it easier to hand off any one of the components to another user.

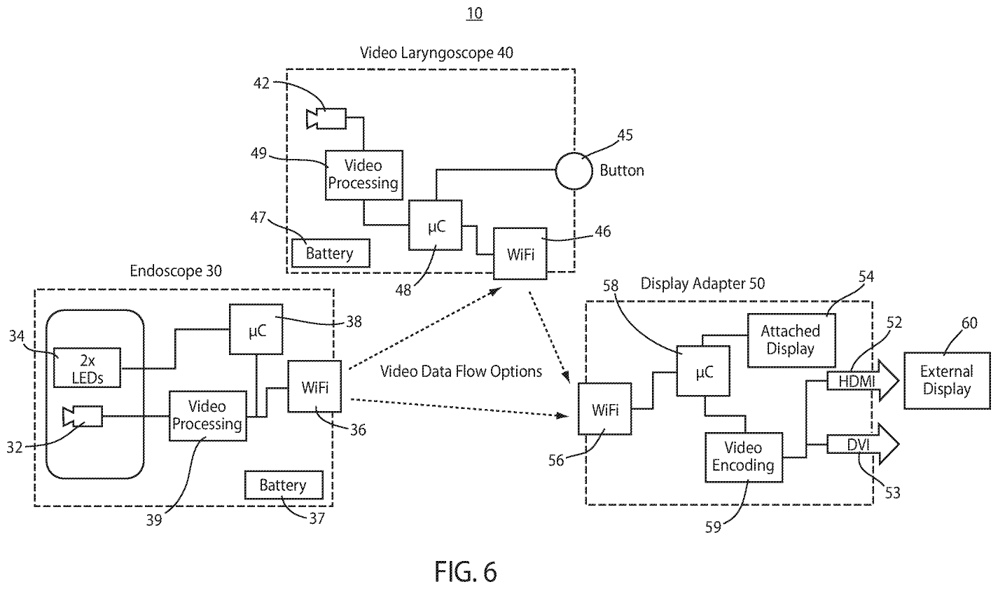

As shown in FIGS. 5 and 6, the endoscope 30 may further include a battery 37, a controller 38, and a video processor 39. The video processor 39 may be provided to process the endoscope video stream 33 output from the endoscope video camera 32 to render it suitable for wireless transmission. The battery 37 may be a single-use battery or a rechargeable battery. The endoscope 30 may be designed as a single-use device in whole or in part. The transmitter 36 may be configured to be removable from the end of the endoscope 30 and may be reusable while the remainder of the endoscope 30 may be disposable after a single use. The video processor 39 may or may not be reusable as well. The battery 37 may be contained within the removable transmitter 36 so as to be recharged between uses. The endoscope 30 may further be configured to have a pull tab or button that enables the endoscope 30 to receive power from the battery 37. The endoscope 30 is paired with receivers of the other components of system 10 and broadcasts live, low-latency video to those receivers. The body of the endoscope 30 is flexible and is configured to be inserted and removed from the inside of the intubation tube 20 as shown in FIG. 1.

As also shown in FIGS. 5 and 6, the laryngoscope 40 may further include a battery 47, a controller 48, and a video processor 49. The video processor 49 may be provided to process the laryngoscope video stream 43 output from the laryngoscope video camera 42 to render it suitable for wireless transmission. The battery 47 may be a single-use battery or a rechargeable battery. According to a first embodiment shown in FIG. 5, the laryngoscope 40 may further include a laryngoscope display 44 for displaying the laryngoscope video stream 43 or the endoscope video stream 33 depending on the selection made by the user by operating the button 45. FIG. 1 shows examples of the different video streams 33 and 43. In this particular embodiment, the transmitter 46 is actually a transceiver that not only transmits the wireless laryngoscope video stream 43, but also may receive the endoscope video stream 33. In the embodiment shown in FIG. 6, the laryngoscope 40 does not include a display. In this case, the user may view the external display 60 connected to the video adaptor 50 via an HDMI port 52 or a DVI port 53, or an attached display 54.

The laryngoscope video camera 42 may be positioned on the blade 41 of the laryngoscope 40 as shown in FIGS. 1 and 2, in order to help with device placement in the patient's airway. The laryngoscope 40 may be reusable except for the blade 41, which would be single-use and replaceable, or a disposable blade cover which would also be single-use and replaceable.

The display adaptor 50 may include a controller 58 and a video processor 59. The video processor 59 may be provided to process the received video streams 33 and 43 to render them suitable for display. The receiver 56 may receive a control signal from transmitter 46 of the laryngoscope 40 representing input received from button 45. The controller 58 would detect the control signal and toggle the streamed video sent to the display(s) 60 and/or 54 to switch between the endoscope video stream 33, the laryngoscope video stream 43, or both simultaneously shown side by side. The display adaptor 50 may be configured with a male plug for insertion into a DVI or HDMI video-IN port on the display 54 or 60. The display adaptor 50 may be connected to a wall power outlet to receive power.

By providing the endoscope video camera 32 (in addition to the laryngoscope video camera 42), a doctor may see what is going on during all stages of intubation, switching to the view that is most helpful at each stage. In addition, the doctor may have orientation information of both video feeds for alignment. Further, the doctor otherwise may have additional information about the airway anatomy, especially in cases considered to be “difficult” by clinical standards.

The endoscope video stream 33 may be configured by the video processor 39 to have visual orientation information relative to the intubation tube 20, the patient anatomy, or the video stream 43 from the laryngoscope 40. The laryngoscope video stream 43 may be configured by the video processor 49 to have visual orientation information relative to the patient anatomy or the video stream 33 from the endoscope 30. This enables a consistent orientation of the video streams viewed by the doctor.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. An intubation system comprising:
an endoscope comprising:
an endoscope video camera and at least one light emitting diode at a distal end, the endoscope video camera generating an endoscope video stream;
a laryngoscope comprising:
a laryngoscope video camera for generating a laryngoscope video stream; and
a button; and
a display adapter configured to receive the endoscope video stream and the laryngoscope video stream and to supply the video stream to a display,
wherein the button provides user selection of which of the laryngoscope video stream, and the endoscope video stream, or both video stream to supply from the display adaptor to the display.

2. The intubation system of claim 1, wherein the endoscope further comprises an endoscope wireless video transmitter on the proximal end for receiving the endoscope video stream from the endoscope video camera and transmitting an endoscope wireless video broadcast.

3. The intubation system of claim 1, wherein the laryngoscope further comprises a laryngoscope wireless video transmitter for receiving the laryngoscope video stream from the laryngoscope video camera and transmitting a laryngoscope wireless video broadcast.

4. The intubation system of claim 2, wherein the laryngoscope further comprises a receiver for receiving the endoscope wireless video broadcast.

5. The intubation system of claim 1, wherein the laryngoscope further comprises a laryngoscope display for displaying at least the laryngoscope video stream.

6. The intubation system of claim 5, wherein the button provides user selection of which of the laryngoscope video stream and the endoscope video stream to display on the laryngoscope display.

7. The intubation system of claim 1 and further comprising an intubation tube having a hollow passageway for receiving the endoscope.

8. The intubation system of claim 1, wherein the endoscope further comprises an illumination source.

9. An intubation system comprising:
an endoscope comprising:
an endoscope video camera and at least one light emitting diode at a distal end; and
an endoscope wireless video transmitter on the proximal end for receiving an endoscope video stream from the endoscope video camera and transmitting an endoscope wireless video broadcast;
a laryngoscope comprising:
a laryngoscope video camera;
a laryngoscope wireless video transmitter for receiving a laryngoscope video stream from the laryngoscope video camera and transmitting a laryngoscope wireless video broadcast; and
a display adapter configured to receive at least one of the endoscope wireless video broadcast and the laryngoscope wireless video broadcast and to supply the video stream transmitted within the wireless video broadcasts to a display.

10. The intubation system of claim 9, wherein the laryngoscope further comprises a button for user selection of which of the laryngoscope video stream and the endoscope video stream to supply from the display adaptor to the display.

11. The intubation system of claim 9, wherein the laryngoscope further comprises a receiver for receiving the endoscope wireless video broadcast.

12. The intubation system of claim 9, wherein the laryngoscope further comprises a laryngoscope display for displaying at least the laryngoscope video stream.

13. The intubation system of claim 12, wherein the laryngoscope further comprises a button for user selection of which of the laryngoscope video stream and the endoscope video stream to display on the laryngoscope display.

14. The intubation system of claim 9 and further comprising an intubation tube having a hollow passageway for receiving the endoscope.

15. The intubation system of claim 9, wherein the endoscope further comprises an illumination source.

16. An intubation system comprising:
an endoscope comprising:
an endoscope video camera and at least one light emitting diode at a distal end, the endoscope video camera generating an endoscope video stream;
a laryngoscope in communication with the endoscope, the laryngoscope comprising:
a laryngoscope video camera for generating a laryngoscope video stream;
a button; and
a laryngoscope display for displaying at least the laryngoscope video stream,
wherein the button allows user selection of which of the laryngoscope video stream and the endoscope video stream to display of the laryngoscope display.

17. The intubation system of claim 16, wherein the endoscope further comprises an endoscope wireless video transmitter on the proximal end for receiving the endoscope video stream from the endoscope video camera and transmitting an endoscope wireless video broadcast.

18. The intubation system of claim 16, wherein the laryngoscope further comprises a laryngoscope wireless video transmitter for receiving the laryngoscope video stream from the laryngoscope video camera and transmitting a laryngoscope wireless video broadcast.

19. The intubation system of claim 17, wherein the laryngoscope further comprises a receiver for receiving the endoscope wireless video broadcast.

20. The intubation system of claim 16 and further comprising an intubation tube having a hollow passageway for receiving the endoscope.
